(19) United States
(12) Patent Application Publication
(10) Pub. No.: US 2012/0226120 A1
(43) Pub. Date: Sep. 6, 2012

(54) CARDIAC MONITORING

(75) Inventors: Johanna Paola Contreras, Boston, MA (US); Harry P. Selker, Wellesley, MA (US)

(73) Assignee: Tufts Medical Center, Inc., Boston, MA (US)

(21) Appl. No.: 13/502,264

(22) PCT Filed: Oct. 19, 2010
(86) PCT No.: PCT/US10/53152

§ 371 Date: May 17, 2012

Related U.S. Application Data
(60) Provisional application No. 61/253,599, filed on Oct. 21, 2009.

(51) Int. Cl. A61B 5/0205; A61B 5/0402; A61N 1/00; A61B 5/1459

(52) U.S. Cl. 600/325

(57) ABSTRACT

A device for monitoring a heart includes a lead wire having a first end and a second end, the second end in contact with tissue of the heart; a first sensor disposed along the length of the lead wire; and a second sensor disposed at the second end of the lead wire. The first sensor is configured to measure an oxygen content of blood in the heart and the second sensor is configured to measure a fluid pressure in the heart. The device further includes a control module connected to the first end of the lead wire and configured to receive signals related to the measured fluid pressure and the measured oxygen content from the first and second sensors.

### CROSS-REFERENCE TO RELATED APPLICATIONS

This application claims priority from U.S. Application No. 61/253,599, filed Oct. 21, 2009, the content of which is incorporated herein by reference in its entirety.

### FIELD OF THE INVENTION

This invention relates to cardiac monitoring.

### BACKGROUND

The characteristic of central mixed venous oxygenation (MvO₂) represents the blood oxygen concentration in blood returning from its cycle around the body and entering the heart to be re-oxygenated. A reduction in MvO₂ is often the earliest and most specific sign of hemodynamic compromise in a patient with advanced cardiopulmonary disease such as heart failure. Insufficient oxygen availability causes organ perfusion failure, lactic acidosis, and, ultimately, liver or kidney failure. The return of MVO₂ to a normal level is a good indicator of an improvement in combined heart and pulmonary function and of a successful therapeutic intervention.

The MvO₂ reflects an integrated function of the cardiac pump function, the pulmonary function as measured by oxygen intake, and other peripheral oxygen demands. The MvO₂ indicates the degree to which the net cardiopulmonary function sufficiently supplies the body's oxygen needs. MvO₂ values below about 30 mmHg suggest that blood returning to the heart is significantly oxygen depleted, an indication of insufficient cardiopulmonary function resulting from a cardiopulmonary disorder such as heart failure or chronic lung disease.

Blood oxygen content (CO₂) represents the total amount of oxygen dissolved in 100 mL of blood and is expressed either as a volume percent or as milliliters of oxygen per deciliter of blood (mL/dL). Blood oxygen content can be measured specifically for arterial blood (CaO₂) and venous blood (CvO₂). Oxygen in blood exists in two forms: dissolved in plasma and carried by hemoglobin. The amount of oxygen dissolved in plasma is calculated by multiplying the oxygen pressure (PO₂) by 0.0031, the solubility coefficient of oxygen in plasma. The amount of oxygen bound to hemoglobin is determined by multiplying 1.38 by the concentration of hemoglobin in the blood by the oxygen saturation in the blood (SO₂), where 1 gram of hemoglobin binds 1.38 mL of oxygen. The total blood oxygen content is thus expressed as

$$\begin{matrix}{CO}_{2} = dissolved {O}_{2} + Hb \text{-} bound {O}_{2} \\ = ( 0.0031 \times {PO}_{2} ) + ( 1.38 \times Hb \times {SO}_{2} )\end{matrix}$$

Since 98% of the oxygen in blood binds to hemoglobin, the oxygen dissolved in plasma can be neglected and the total blood oxygen content can be approximated as only the amount of hemoglobin-bound oxygen. Blood oxygen content is thus highly dependent on hemoglobin concentration and oxygen saturation.

Given the oxygen saturation and hemoglobin concentration, blood oxygen content can be determined. For instance, a normal concentration of hemoglobin is 15 grams per 100 mL of blood. Using the above equation and assuming a normal arterial oxygen saturation (SaO₂) of 97%, the arterial blood oxygen content (CaO₂) is determined to be 20.1 vol. %. On the venous side, normal venous oxygen saturation (SvO₂) is 75%, giving a CvO₂ of 15.5 vol. %. That is, tissues normally use 25% of the oxygen delivered to them and return 75% of the oxygen back to the lungs. The arterial-venous oxygen content difference, or oxygen extraction, is 5 vol. %.

Oxygen transport and delivery (DO₂) represents the amount of oxygen delivered to the tissues and is measured in mL or in mL per minute. Oxygen transport is dependent upon two factors: the ability of the heart to maintain an adequate blood flow to the tissues (i.e., cardiac output), and the ability of the lungs to oxygenate blood as the blood passes through the pulmonary capillary network. The latter factor is reflected in the CaO₂ level. Oxygen transport is determined from the following expression:

O2 transport=Cardiac Output (CO)×Oxygen Content (CO₂)×10

The factor of 10 converts oxygen content to milliliters of oxygen per minute.

For instance, continuing with the above example, for a normal cardiac output of 5 Liters per minute, arterial oxygen transport (i.e., total oxygen delivery) is 1005 mL of oxygen per minute. Venous oxygen transport, or the amount of unused oxygen returning to the heart, is 775 mL of oxygen per minute.

When the balance between oxygen supply and oxygen demand is threatened, the body mobilizes its compensatory mechanism to ensure adequate oxygen availability by increasing cardiac output and/or increasing oxygen extraction from the blood. If cardiac output falls (e.g., due to a cardiopulmonary disorder), one of these two compensatory mechanisms is eliminated. If, however, blood oxygen content is reduced due to a decrease in SaO₂ or in hemoglobin concentration, both compensatory mechanisms remain available, albeit less efficient. A patient is thus less able to tolerate a drop in cardiac output than a decrease in SaO₂ or hemoglobin concentration.

Normal values for the mixed venous oxygen saturation (SvO₂) range from 60% to 80%. An SvO₂ value around 50% to 60% indicates a mild decrease in the mixed venous oxygen reserve. For SvO₂ values less than 50%, significant depletion of the mixed venous reserve reduces the patient's capacity to buffer hypoxic threat. At SvO₂ values less than 32%, a minimum mixed venous saturation has been reached. Anaerobic metabolism and lactic acidosis quickly follow and there is a risk of organ damage and/or circulatory collapse. Low SvO₂ values are generally caused by cardiovascular insufficiency, increased oxygen demand, hypoxemia, anemia, and/or active hemorrhage. The physiologic tolerance of a patient to a fall in SvO₂ and the time to rebound to the patient's baseline SvO₂ level depend on a variety of factors, including the underlying cause of the decrease, the magnitude of the decrease, the rapidity of institution and the effectiveness of corrective therapies, and the patient's baseline (i.e., steady state) SvO₂ and cardiac reserves.

In the opposite direction, SvO₂ values greater than 80% cause decreased cellular oxygen uptake and/or utilization. Causes of a high SvO₂ include intracardial or intravascular shunts (common in sepsis and cirrhosis); increased affinity of hemoglobin for oxygen (due, for instance, to alkalemia, hypocarbia, hypothermia, or administration of a large amount of banked blood); cytotoxicity (e.g., ethanol toxicity, cyanide poisoning, or sepsis); hypometabolism (hypothermia); polycythemia, or muscle paralysis (e.g., due to a neuromuscular blocking agent).

### SUMMARY

In a general aspect, a device for monitoring a heart includes a lead wire having a first end and a second end, the second end in contact with tissue of the heart; a first sensor disposed along the length of the lead wire; and a second sensor disposed at the second end of the lead wire. The first sensor is configured to measure an oxygen content of blood in the heart and the second sensor is configured to measure a fluid pressure in the heart. The device further includes a control module connected to the first end of the lead wire and configured to receive signals related to the measured fluid pressure and the measured oxygen content from the first and second sensors.

Embodiments may include one or more of the following. The first sensor and the second sensor are configured to operate simultaneously. The second sensor is configured to continuously measure the fluid pressure. The first sensor is configured to continuously measure the oxygen content of blood in the heart.

The first sensor and the second sensor are positioned in a right ventricle of the heart. The second sensor is configured to measure the fluid pressure in the right ventricle and first sensor is configured to measure the oxygen content of blood in the right ventricle.

The second sensor is further configured to measure a pulse pressure in the heart, an intracardiac electrocardiogram, or an impedance of tissue in the heart. The impedance of tissue in the heart is indicative of the fluid pressure in the heart. The sensor is further configured to measure an impedance of pulmonary tissue. The second sensor includes a pressure sensitive membrane. The pressure sensitive membrane is formed of titanium. The second sensor includes electrodes disposed on an external surface of the sensor.

The first sensor includes an optical device configured to emit light and to detect an amount of light reflected by the blood in the heart. The optical device is a fiber-optic device. The amount of light reflected by the blood in the heart is indicative of the oxygen content of blood in the heart.

The device further includes a pacemaker. The second end of the lead wire is a lead of the pacemaker. The second end of the lead wire is mechanically anchored to tissue of a right ventricle of the heart. The device further includes a second lead wire, a first end of the second lead wire connected to the control module. A second end of the second lead wire is a lead of the pacemaker. The second end of the second lead wire is mechanically anchored to tissue of a right atrium of the heart. The control module configured to control the pacemaker in part on the basis of the received signals related to the measured oxygen content.

The first sensor and the second sensor are configured to remain in the heart for up to 10 years. The control module includes a communication module and a power supply. The communication module is a wireless communication module.

A cardiac monitoring device as described herein has a number of advantages. As cardiopulmonary disease, such as chronic cardiopulmonary failure, progresses in a patient, monitoring of the volume and hemodynamic status of the patient becomes more challenging due to the wide range of clinical manifestations and patient-driven characteristics associated with the disease. An implantable sensor system that continuously and remotely monitors blood oxygen content and fluid pressure in real time simplifies patient monitoring by enabling early detection of organ perfusion and the degree of compensation or decompensation in a patient. The data collected by the sensor help to guide therapy, triage, and cost-effective long term management of the patient, including when the patient is at a location remote from the medical staff supervising care.

Continuous, remote SvO₂ and pressure monitoring improves not only the therapeutic effects of treatment, but also the quality of life of the patient. Wireless communication between the sensor and a remote computer means that the patient is not attached to an intravenous (IV) line but rather is allowed move about freely. Monitoring can continue at home rather than in a hospital setting, saving money and reducing inconvenience for the patient. Additionally, with the availability of early indications of worsening of heart failure reflected by dropping MvO₂ levels, treatment can be instituted and/or adjusted, thus preventing further worsening. This treatment allows better health to be maintained and avoids the potentially severe discomfort and disability of worsening cardiopulmonary function. This treatment also significantly reduces the risk of systemic infection that comes with the placement of multiple Swan-Ganz catheters, an intervention that is routinely used today as a last resort to obtain an adequate hemodynamic assessment on patients in heart failure. Many patients with heart failure, pulmonary hypertension, or other types of cardiopulmonary disease are or will become candidates for an implantable therapeutic device, such as a prophylactic implantable cardioverter defibrillator (ICD), a biventricular ICD (BivICD), or a permanent pacemaker (PPM). The integration of a SvO₂ and fluid pressure biosensor with another implanted device is convenient, cost effective, and minimizes invasive medical procedures. Furthermore, the data measured by the sensor can be used to improve the performance of the therapeutic device. For instance, when a SvO₂ and fluid pressure biosensor is incorporated with a PPM or an ICD, the PPM/ICD software is modified to use SvO₂ in an algorithm for setting the pacing rate in order to avoid over-pacing, which can be associated with worsening of patient hemodynamic status and increased morbidity and mortality. The inclusion of SvO₂ readings in the therapeutic algorithm provides additional information to the pacemaker regarding overall patient hemodynamics and increases the accuracy of the therapeutic algorithms. Thus overpacing and use of inappropriate ICD defibrillator shock therapy can be decreased or eliminated. A reduction in such therapies in turn minimizes the deleterious electrophysiologic impact of the pacemaker and will have a major impact in improvement of patient's quality of life.

Other features and advantages of the invention are apparent from the following description and from the claims.

### BRIEF DESCRIPTION OF DRAWINGS

FIG. 1 is a schematic diagram of a cardiac sensor system implanted in the right ventricle of a heart.

FIG. 2 is a block diagram of the control module of the cardiac sensor system of FIG. 1.

FIG. 3 is a schematic diagram of a cardiac sensor system integrated with a pacemaker.

FIG. 4 is a block diagram of the pulse generator associated with the sensor system and pacemaker of FIG. 3.

### DETAILED DESCRIPTION

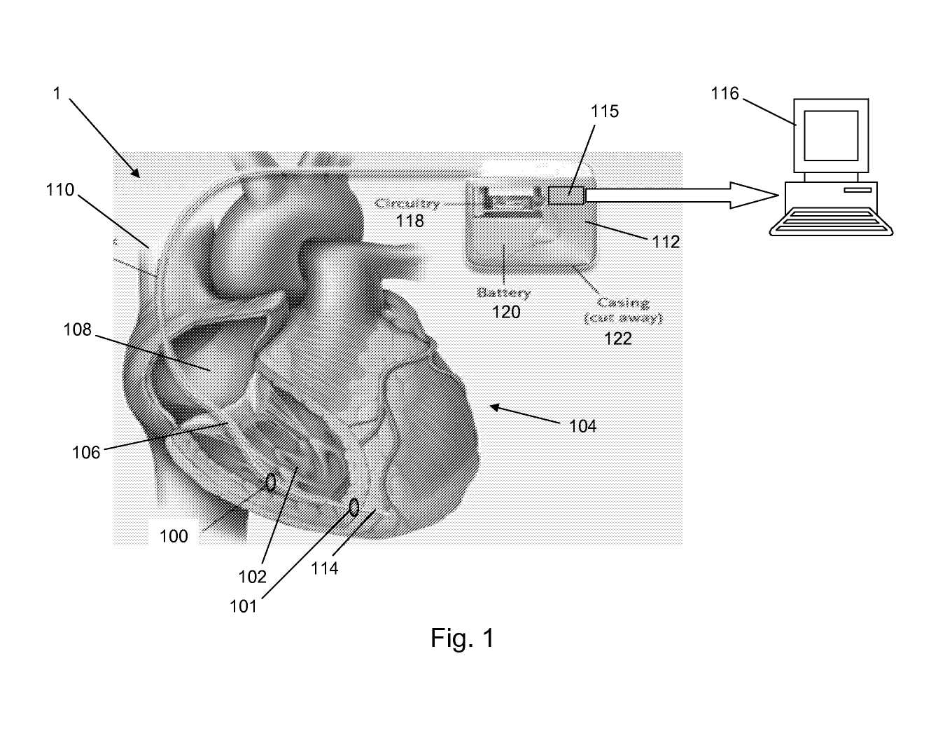

Referring to FIG. 1, a sensor system 1 implanted in a right ventricle 102 of a heart 104 continuously monitors physiological parameters of a patient. An oxygen sensor 100 measures the central mixed venous oxygen level (MvO₂) or the central mixed venous oxygen saturation (SvO₂) and the percent oxygen saturation in right ventricular blood. Simultaneously, a pressure sensor 101 measures the central venous fluid pressure and pulse pressure in the right ventricle and the maximum positive and negative rate of change of the pressure during the cardiac cycle (dP/dt). In some embodiments, pressure sensor 101 also measures an intracardiac electrocardiogram and an impedance of heart and lung tissue. In other embodiments, only oxygen sensor 100 is used. The use of an oxygen sensor alone is useful, for instance, for monitoring patients with pulmonary hypertension.

These physiological parameters provide data that can be used to identify and monitor organ perfusion, congestion in the chest cavity, and the degree of compensation or decompensation in patients with chronic cardiopulmonary failure or other types of cardiopulmonary disease. When coupled with cardiac output measurements, these data enable the calculation of oxygen transport and oxygen consumption; early identification of impending or actual global tissue hypoxia; a determination of the cause of a hypoxic episode; an assessment of the response of a patient to a treatment of hypoxia; and a prediction of patient survival based on an underlying cause of a hypoxia episode and on the patient's response to the hypoxia treatment.

Oxygen sensor 100 is approximately less than 1 cm in diameter and is positioned along the length of a lead 106 that passes through a right atrium 108 and a superior vena cava 110 at about 3-4 cm above the tip of the lead. Pressure sensor 101 is positioned toward the end of lead 106, embedded in the wall of the right ventricle towards the apical septum 102. Lead 106 and sensor 100 are inserted intravenously into the right ventricle through the subclavian or cephalic vein of the patient. Lead 106 connects to a control module 112 positioned in a subcutaneous device pocket in the subclavicular region of the patient, which pocket is formed by a small cutaneous incision, as in currently performed during the implantation of a pacemaker. Lead 106 is between 5-7 mm thick, and is typically about 5 mm thick.

A tip of 114 of lead 106 is anchored in the myocardium of heart 104 by soft tines or a tiny screw (not shown). A steroid elutes from tip 114 to decrease inflammation at the tip-myocardium interface, thus improving the chronicity of sensor system 1. As a result, the sensor system 1 is able to remain implanted for long periods of time, allowing long term monitoring of physiological parameters.

Measurement data are transmitted from oxygen sensor 100 and pressure sensor 101 to control module 112 along lead 106. Control module 112 includes a wireless communication module 115, such as an antenna coil. Communication module 115 wirelessly communicates the measurement data to a remote computer 116 for display, storage, or processing. Computer 116 may be, for instance, a clinician's computer, a patient's computer, or a handheld computing device. Communication between control module 108 and computer 112 may be periodic or upon request by computer 112. For instance, computer 112 may calculate both a continuous SvO₂ level and an average SvO₂ level at a preselected timing interval. Also, once a baseline SvO₂ of the patient is obtained, an alarm setting can be programmed that will be activated at pretermined levels of SVO₂, thus allowing early recognition of a decline or a decompensated status.

Control module 108 also includes control circuitry 118 that controls the operation of sensor 100 and communication module 115. A lithium battery 120 in control module 112 supplies power to control circuitry 118, communication module 115, and sensor 100. The lifetime of battery 120 is typically in the range of 5-10 years and depends on factors such as the output voltage of control module 112, the resistance of lead 106 and sensor 100, and the frequency and duration of use of the battery. The components in control module 108 are enclosed in a biocompatible casing 122.

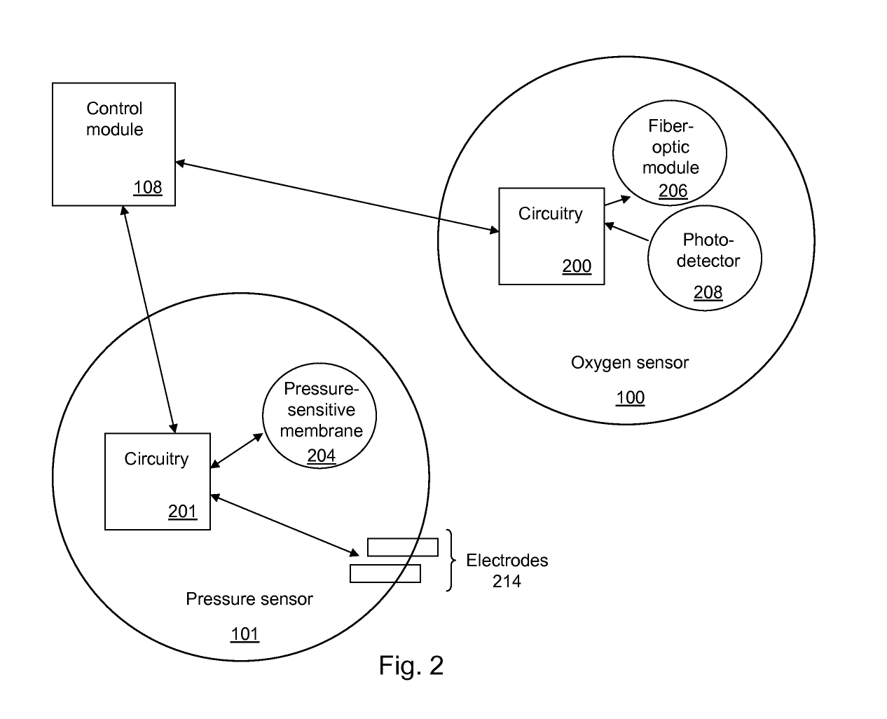

Referring to FIG. 2, oxygen sensor 100 and pressure sensor 101 are hermetically sealed devices made of titanium, iridium, or another biocompatible material that is pharmacologically inert, nontoxic, sterilizable, and able to function in the environmental conditions of the body. Ideally the material is not affected by stress cracking or metal ion oxidation. Circuitry 200 in sensor 100 and circuitry 201 in sensor 101 control the operation of measurement devices housed in sensors 100 and 101 and control the communication between the sensors and control module 112.

A light emission module 206 in oxygen sensor 100 includes a red (660 nm) and/or infrared (880 nm) light emitting diode (LED) hermetically sealed in a sapphire capsule. The LED emits light which illuminates blood in the right ventricle. The amount of light reflected by the blood, which is indicative of the oxygen saturation (i.e., the SvO₂) is detected by a photodetector 208.

A titanium pressure sensing membrane 204 mounted on pressure sensor 101 measures fluid pressure and pulse pressure in the right ventricle or right atrium.

A set of electrodes 214 mounted on the external surface of pressure sensor 101 measures the impedance of tissue in the chest cavity, such as cardiac tissue and pulmonary tissue, at a digital rate of 128 Hz. Impedance measurements allow for portioned analysis of contractile cardiac function and pulmonary ventilation function. Average pulmonary impedance, e.g., averaged over a period of 72 hours or more, provides a baseline value against which an instantaneous impedance measurement can be compared. Signal processing of the impedance data allows deviations from baseline impedance values to be detected. For instance, a decrease in lung impedance is indicative of increasing fluid content and congestion in the lungs, which can lead to congestive heart failure.

In some embodiments, the sensor system is integrated with another implantable diagnostic or therapeutic device, such as a prophylactic implantable cardioverter defibrillator (ICD), a biventricular ICD, or a permanent pacemaker (PPM). In general, when the sensor system is integrated with another implantable device, certain structures (e.g., lead 106 in FIG. 1) may be shared between either or both of sensor 100 or sensor 101 and the other implantable device.

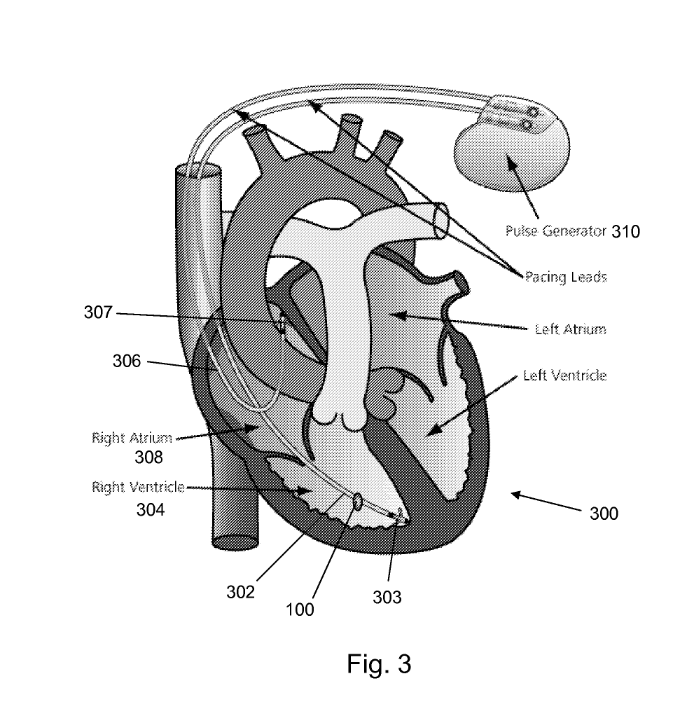

Referring to FIG. 3, a sensor system 300 is combined with a pacemaker and implanted in a heart 300. Oxygen sensor 100 is positioned along a ventricular lead 302 of the pacemaker; pressure sensor 101 is positioned at the end of the lead 302. Ventricular lead 302 is anchored in the myocardium of a right ventricle 304 by an anchor 303. An atrial lead 306 of the pacemaker is anchored in the myocardium of a right atrium towards the right interatrial septum 308 by an anchor 307. In some instances, a sensor system such as that shown in FIG. 1 is later upgraded to include a pacemaker (i.e., to become sensor system 300) if a patient's illness evolves to indicate the use of a pacemaker. In other instances, an existing pacemaker is upgraded to include sensors 100 and 101.

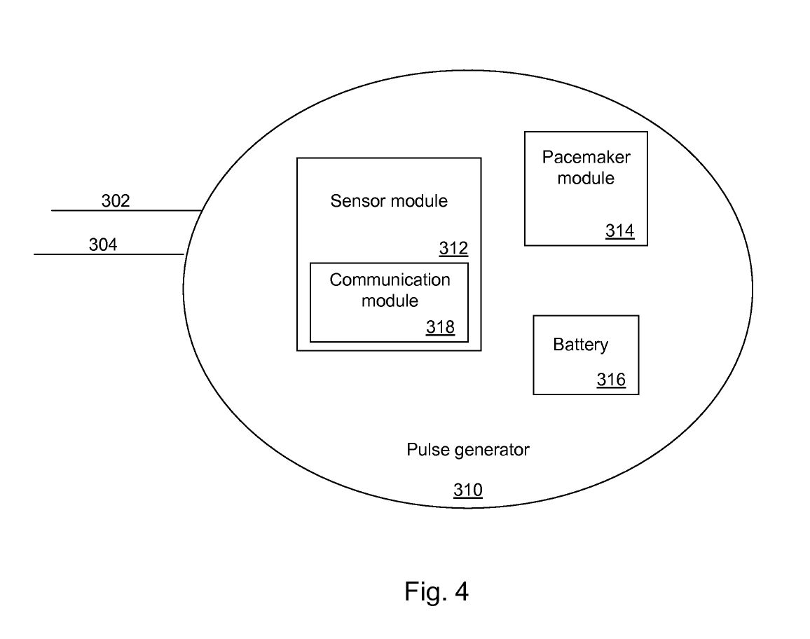

In some embodiments, pressure sensor 101 is positioned at the end of atrial lead 306. In some instances, atrial lead 306 is directed toward the base of the inter-atrial septum (not shown) such that pressure sensor 101 is embedded in the wall of the right atrium. The measurements of the right atrial pressure provided by the pressure sensor located on the right atrial lead generally are more accurate than measurements of the right ventricular pressure provided by a pressure sensor located on a right ventricular lead (e.g., sensor 101 in FIG. 1). The placement of both an atrial lead and a ventricular lead is a more invasive procedure (such as a transseptal puncture) than the placement of only a ventricular lead. However, when the sensor system is used in conjunction with a pacemaker (e.g., a dual chamber pacemaker, a PPM/ICD, or a BivICD), an atrial lead and a ventricular lead are already used and thus no additional intervention occurs. Referring to FIGS. 3 and 4, a pulse generator 310 is implanted in a subcutaneous device pocket in the subcutaneous region of a patient and connects to ventricular lead 302 and atrial lead 306. Pulse generator 310 includes a sensor module 312, a pacemaker module 314, and a lithium battery 316. Sensor module 312 controls the operation of sensors 100 and 101 and receives measurement data from sensors 100 and 101 via lead 302. Sensor module 312 includes a wireless communication module 318 that communicates the measurement data to a remote computer. Pacemaker module 314 sends electrical pacing signals along ventricular lead 302 and atrial lead 306. Pacemaker module 314 controls the pacing of the pacemaker according to a predetermined algorithm that takes into account physiological parameters including heart rate, QRS duration and morphology, PR intervals, and SvO₂ levels. Battery 316 provides power to sensor module 312, pacemaker module 314, communication module 318, and sensors 100 and 101. The lifetime of battery 316 is typically 5-10 years and depends on factors such as the output voltage of sensor module 312 and pacemaker module 314, the resistance of leads 302 and 306 and sensor 100, the pacing rate, and the frequency and duration of use of the battery.

In some embodiments, a sensor system that performs continuous SvO₂ and pressure monitoring is used in conjunction with diagnostic and/or treatment algorithms that enable more accurate monitoring, diagnosis and treatment. In some instances, algorithms incorporated into the control module enable remote management of patients. When the sensor system is incorporated with a pacemaker, the algorithms also enable more accurate and efficient pacing. Such systems can be used for a variety of applications, including the following:
Guiding outpatient treatment and monitoring of chronic cardiac, pulmonary, or muscle failure
Facilitating the early identification of patients with acute cardiopulmonary decompensation that will benefit from early hospitalization and/or treatment modification
Enabling follow up to medical therapy and observation of a patient's response to changes in therapy in both inpatient and outpatient settings
Guiding treatment and titration of continuous-inotrope-based home treatment on patients waiting for cardiac, lung or combined lung cardiac transplant
Guiding treatment of advanced cardiomyopathy, advanced pulmonary disease, or end-stage muscular disease
Guiding treatment of moderate to severe primary or secondary pulmonary hypertension
Monitoring of a patient with chronic pulmonary hypertension undergoing treatment with continuous IV vasodilator, prostaglandin or immune-modulating agent therapy
Monitoring and guiding treatment of advanced pulmonary fibrosis, emphysema, or interstitial lung and bronchospastic disease
Guiding the management of a patient on mechanical ventilation or the weaning of a patient from mechanical ventilation, including patients for whom weaning by other methods has failed
Facilitating acute post-surgical care
Monitoring high-risk surgical anesthesia and post-surgical care
Guiding the assessment and/or adjustment of therapy and routine nursing care
Guiding the management of a patient having intra-aortic balloon counterpulsation
Guiding the management of a patient with a left, right, or biventricular assisted device to ensure hemodynamic stability
Facilitating the identification of patients who have life-threatening arrhythmias with major hemodynamic manifestations
Guiding the management of a patient after an acute cerebrovascular accident or seizure
Guiding the early identification of vegetative patients that could potentially become donors for organ transplantation

It is to be understood that the foregoing description is intended to illustrate and not to limit the scope of the invention, which is defined by the scope of the appended claims. Other embodiments are within the scope of the following claims.

What is claimed is:

1. A device for monitoring a heart, the device comprising:
a lead wire having a first end and a second end, the second end in contact with tissue of the heart;
a first sensor disposed along the length of the lead wire and configured to measure an oxygen content of blood in the heart a second sensor disposed at the second end of the lead wire and configured to measure a fluid pressure in the heart or at the end of the atrial lead; and
a control module connected to the first end of the lead wire and configured to to receive signals related to the measured fluid pressure and the measured oxygen content from the first and second sensors.

2. The device of claim 1, wherein the first sensor and the second sensor are configured to operate simultaneously.

3. The device of claim 1, wherein the first sensor is configured to continuously measure the oxygen content of blood in the heart and the second sensor is configured to continuously measure the fluid pressure.

4. The device of claim 1, wherein the first sensor and the second sensor are positioned in a right ventricle of the heart.

5. The device of claim 4, wherein the second sensor is configured to measure the fluid pressure in the right ventricle and first sensor is configured to measure the oxygen content of blood in the right ventricle.

6. The device of claim 1, wherein the second sensor is further configured to measure a pulse pressure in the heart.

7. The device of claim 1, wherein the second sensor is further configured to measure an intracardiac electrocardiogram.

8. The device of claim 1, wherein the second sensor is further configured to measure an impedance of tissue in the heart.

9. The device of claim 8, wherein the sensor is further configured to measure an impedance of pulmonary tissue.

10. The device of claim 1, wherein the first sensor comprises an optical device configured to emit light and to detect an amount of light reflected by the blood in the heart.

11. The device of claim 10, wherein the amount of light reflected by the blood in the heart is indicative of the oxygen content of blood in the heart.

12. The device of claim 1, wherein the second sensor comprises a pressure sensitive membrane.

13. The device of claim 1, wherein the second sensor comprises electrodes disposed on an external surface of the sensor.

14. The device of claim 1, further comprising a pacemaker.

15. The device of claim 14, wherein the second end of the lead wire is a lead of the pacemaker.

16. The device of claim 15, wherein the second end of the lead wire is mechanically anchored to tissue of a right ventricle of the heart.

17. The device of claim 14, further comprising a second lead wire, a first end of the second lead wire connected to the control module, wherein a second end of the second lead wire is a lead of the pacemaker.

18. The device of claim 14, wherein the control module configured to control the pacemaker in part on the basis of the received signals related to the measured oxygen content.

19. The device of claim 1, wherein the first sensor and the second sensor are configured to remain in the heart for up to 10 years.

20. The device of claim 1, wherein the control module includes a wireless communication module.
